(19) United States
(12) Patent Application Publication
(10) Pub. No.: US 2016/0158473 A1
(43) Pub. Date: Jun. 9, 2016

(54) Ventilator circuit stabilizer and method of stabilizing a ventilator

(71) Applicants: Michael Schuster, West Chester, PA (US); Gregory E. Doll, Bethesda, MD (US)

(72) Inventors: Michael Schuster, West Chester, PA (US); Gregory E. Doll, Bethesda, MD (US)

(21) Appl. No.: 14/937,947

(22) Filed: Nov. 11, 2015

Related U.S. Application Data
(60) Provisional application No. 62/087,580, filed on Dec. 4, 2014.
(60) Provisional application No. 62/103,597, filed on Jan. 15, 2015.

(51) Int. Cl. A61M 16/04; A61M 25/02

(52) U.S. Cl. CPC A61M 16/0465; A61M 16/0497; A61M 25/02; A61M 2025/0206; A61M 2025/026

(57) ABSTRACT

Provided are a device and method for securing the ventilator tubing circuitry to a patient so as to prevent the circuitry from becoming disconnected from the patient or ventilator or becoming entangled, crushed or otherwise made non-functional thereby causing possible harm to the patient.

### FIELD OF THE INVENTION

The present invention relates to securing the ventilator tubing circuitry to a patient so as to prevent the circuitry from becoming disconnected from the patient (or ventilator) or becoming entangled, crushed or otherwise made non-functional thereby causing possible harm to the patient.

### BACKGROUND OF THE INVENTION

Patients who are unable to breath on their own typically have a tracheostomy or endotracheal tube placed in their trachea. A ventilator tubing circuit is attached to the tube on one end and a ventilator on the opposite end. The tracheostomy tube or endotracheal tube are typically attached to the patient with a securement device that keeps the tube from being displaced from the patients trachea.

U.S. Pat. No. 5,672,159 (Warrick), issued Sep. 30, 1997, the complete disclosure of which is incorporated herein by reference, discloses a device to support and secure a ventilator tubing to a patient that is rather cumbersome and restrictive for the patient to both attach and wear. Because the patients' health is already extremely compromised anything that limits or restricts movement will cause exponential negative outcomes. The product is restrictive as well as more difficult to put on the patient. It is also complicated for a caregiver to properly secure a device. The device is worn around the shoulder thereby keeping the circuitry off to the side where, with mobile patients (wheelchair, motorized scooter, etc.), it increases the probability of allowing the circuitry to fall away from the mobility device and become trapped, stuck or crushed under the wheels of the mobility device.

U.S. Pat. No. 5,244,464 (Madden), issued Sep. 14, 1993, discloses a device that traps the circuitry between a main strap and a completely releaseable secondary holding strap which can be completely disconnected from the main strap allowing for the circuitry to become unsecured and increasing the concern for patient safety.

There is a need for a device to assist in ensuring that the ventilator tubing itself does not fall away from the patient thereby allowing it to be damaged to the extent that the oxygen from the ventilator can no longer pass freely through the tubing to the patient, thereby causing significant or permanent injury to the patient.

### SUMMARY OF THE INVENTION

It is an object of this invention to provide a product that was comfortable for a patient while providing the security necessary to keep the ventilator tubing from being compromised and that is easy for a caregiver to apply and remove.

Objects of the invention can be obtained by a ventilator circuit stabilizer comprising:
a torso strap sized and constructed to encircle a patient's torso, the torso strap comprising an elongated foam material having a Velcro loop material mounted on a loop side and a soft material suitable for application to human skin on an opposing soft side, and a Velcro hook is mounted on the soft side of the torso strap at a first end of the torso strap; and
a ventilator strap sized and constructed to fasten a ventilator circuit to the torso strap, the ventilator strap comprising a foam material having Velcro loop material mounted to a loop side and a soft material suitable for application to skin mounted on an opposing soft side, a Velcro hook is mounted on the soft side of the ventilator strap and at a first end of the ventilator strap, a second end of the ventilator strap is mounted loop side down to the loop side of torso strap near the first end of torso strap.

Objects of the invention can also be obtained by a method of fastening a ventilator circuit stabilizer to a patient comprising:
providing a torso strap sized and constructed to encircle a patient's torso, the torso strap comprising an elongated foam material having a Velcro loop material mounted on a loop side and a soft material suitable for application to human skin on an opposing soft side, and a Velcro hook is mounted on the soft side of the torso strap at a first end of the torso strap, and a ventilator strap sized and constructed to fasten a ventilator circuit to the torso strap, the ventilator strap comprising a foam material having Velcro loop material mounted to a loop side and a soft material suitable for application to skin mounted on an opposing soft side, a Velcro hook is mounted on the soft side of the ventilator strap and at a first end of the ventilator strap, a second end of the ventilator strap is mounted loop side down to the loop side of torso strap near the first end of torso strap;
encircling a patient torso with the torso strap with the soft side of the torso strap facing a skin of the patient;
fastening the torso strap by pressing the Velcro hook of the torso strap into the loop material on the torso strap;
laying a ventilator circuit stabilizer across the fastened torso strap;
moving the first end of the stabilizer strap over the ventilator circuit stabilizer; and
fastening the Velcro hook of the ventilator strap to the loop material of the torso strap.

### BRIEF DESCRIPTION OF THE DRAWINGS

FIG. 1 illustrates a view of a ventilator circuit stabilizer.

FIG. 2 illustrates a view of a ventilator circuit stabilizer fastened to a patient.

FIG. 3 illustrates a view of a ventilator circuit stabilizer.

FIG. 4 illustrates a view of a ventilator circuit stabilizer fastened to a patient.

### DETAILED DESCRIPTION OF THE INVENTION AND USE

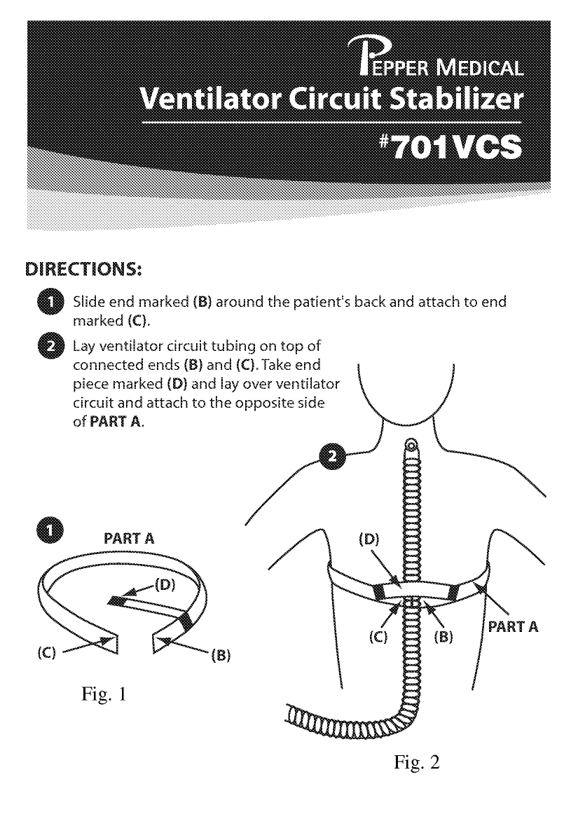

The invention will be explained with reference to the attached non-limiting FIGS. FIG. 1 shows a view of an example of an unmounted ventilator circuit stabilizer. FIG. 2 shows the ventilator circuit stabilizer fastened to a patient.

The stabilizer comprises a torso strap (part A), comprising a foam material covered with Velcro loop material on a loop side and a soft material suitable for application to skin on the opposing soft side. An example of the soft material is cotton. The length of the torso strap (A) varies depending on patient type: infant, pediatric, adult, bariatric, etc. The torso strap (A) must be of sufficient length to encircle the patient's torso. A piece of Velcro hook is mounted, for example welded, to a first end of the soft side of the torso strap, shown at (B). A second end of the torso strap (A) is shown at (C)

The stabilizer further comprises a ventilator strap (part D) that is sized and constructed to mount the ventilator circuit to the torso strap (A). In a preferred embodiment, the ventilator strap (A) comprises foam material covered with Velcro loop material on a loop side and a soft material suitable for application to skin on the opposing soft side. An example of the soft material is cotton. The ventilator strap (D) has a Velcro hook mounted, such as welded, to a first end of the soft side of the ventilator strap (D). The second end of the ventilator strap (D) is mounted loop side down to the loop side of torso strap (A) near the first end (B) of the torso strap (A). Preferably, the second end of the ventilator strap (D) is mounted to the first end (B) of the torso strap (A) where the Velcro hook is mounted.

The torso strap (A) can then be looped around the patient's torso like a belt with the soft side facing the patient. The first end (B) with Velcro hook can be removably attached in the front of the patient to the opposite side of the torso strap (A) (Velcro loop) by pressing the Velcro hook to the Velcro loop. This will keep the ventilator circuit tubing securely attached to the patient and since the Velcro hook at the first end (B) can be mounted in different locations the torso strap (A) can be secured around the torso.

The ventilator circuit is then placed on top of the front of the torso strap (A) and the first end of the ventilator strap (D) is moved over the ventilator circuit so that the ventilator strap is laid over the ventilator circuit. The first end of the ventilator strap (D) is then secured to the torso strap (A) by pressing the Velcro hook of the ventilator strap (D) onto the Velcro loop of the torso strap (A). This will secure the ventilator circuit to the patient and prevent the ventilator circuit from falling away from the patient's body.

The stabilizer secures the ventilator circuit to the patient's torso in such a manner that stabilizer will not allow the circuitry to fall away from the patient when the patient moves and prevents the circuitry from interfering when a clinician is assisting a patient with other clinical needs. The stabilizer also allows a clinician/caregiver to move the ventilator circuitry to either side of a patient if the clinician/caregiver needs to do clinical assessment of the area where the ventilator circuitry rests on the patient.

A specific non-limiting example of the torso strap (A) is a 34 inch to 60 inch×1.5 inch length of white foam with loop material laminated to one side of the foam and cotton fabric laminated to the opposite soft side of the foam. Attached to the first end (B) of the torso strap on the loop side is a 2 inch×0.5 inch piece of HTH hook material. The hook material can be welded at both ends (B) (C) to the torso strap (A). The hook acts as the fastening element that secures the torso strap around the patient's torso.

A specific non-limiting example of the ventilator strap (D) is a 5 inch×0.5 inch length of blue foam with loop material laminated to one side of the foam and cotton fabric laminated to the opposite side of the foam. A piece of HTH hook, 1.75 inch×0.5 inch is welded to the first end of the loop material side of Part B. The second end of the ventilator strap is welded, loop material side down, to the cotton side of the torso strap (D), 2 inches from the first end of the torso strap where the HTH hook is welded to the loop material side of the torso strap. The ventilator strap (D) secures the ventilator circuitry to the torso strap.

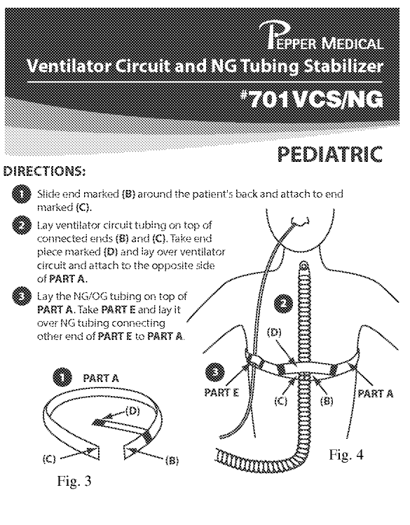

FIGS. 3 and 4 show another embodiment of the invention. Attached to the torso strap (A) is at least one securement strap (E), similar in shape to the securement strap (D). The securement strap (E) can be shorter in length than the securement strap (D). The purpose of the securement strap (E) is to provide securement for tubes, catheters or other medical devices that should be secured for safety purposes and to enable clinical staff to maneuver around a patient without concern for disconnecting the tubes, catheters or other medical devices. Some, but not all, examples of other devices that would benefit from the securement strap (E) would be, naso-gastric feeding tubes, oral-gastric feeding tubes central IV lines. More than one securement straps (E) can be attached to the torso strap (A). The securement strap (E) can have HTH Hook attached at both ends for fastening to the torso strap (A). One end of the securement strap (E) can be permanently attached to the torso strap (A) if desired.

While the claimed invention has been described in detail and with reference to specific embodiments thereof, it will be apparent to one of ordinary skill in the art that various changes and modifications can be made to the claimed invention without departing from the spirit and scope thereof.

1. A ventilator circuit stabilizer comprising:
a torso strap sized and constructed to encircle a patient's torso, the torso strap comprising an elongated foam material having a Velcro loop material mounted on a loop side and a soft material suitable for application to human skin on an opposing soft side, and a Velcro hook is mounted on the soft side of the torso strap at a first end of the torso strap; and
a ventilator strap sized and constructed to fasten a ventilator circuit to the torso strap, the ventilator strap comprising a foam material having Velcro loop material mounted to a loop side and a soft material suitable for application to skin mounted on an opposing soft side, a Velcro hook is mounted on the soft side of the ventilator strap and at a first end of the ventilator strap, a second end of the ventilator strap is mounted loop side down to the loop side of torso strap near the first end of torso strap.

2. The ventilator circuit stabilizer according to claim 1, wherein the soft material comprises cotton.

3. The ventilator circuit stabilizer according to claim 1, further comprising at least one securement strap comprising a Velcro hook for attaching the securement strap to the torso strap and being constructed for securing medical devices to the patient.

4. The ventilator circuit stabilizer according to claim 3, wherein the medical device comprises tubes or catheters.

5. A method of fastening a ventilator circuit stabilizer to a patient comprising:
providing a torso strap sized and constructed to encircle a patient's torso, the torso strap comprising an elongated foam material having a Velcro loop material mounted on a loop side and a soft material suitable for application to human skin on an opposing soft side, and a Velcro hook is mounted on the soft side of the torso strap at a first end of the torso strap, and a ventilator strap sized and constructed to fasten a ventilator circuit to the torso strap, the ventilator strap comprising a foam material having Velcro loop material mounted to a loop side and a soft material suitable for application to skin mounted on an opposing soft side, a Velcro hook is mounted on the soft side of the ventilator strap and at a first end of the ventilator strap, a second end of the ventilator strap is mounted loop side down to the loop side of torso strap near the first end of torso strap;
encircling a patient torso with the torso strap with the soft side of the torso strap facing a skin of the patient;
fastening the torso strap by pressing the Velcro hook of the torso strap into the loop material on the torso strap;
laying a ventilator circuit stabilizer across the fastened torso strap;
moving the first end of the stabilizer strap over the ventilator circuit stabilizer; and
fastening the Velcro hook of the ventilator strap to the loop material of the torso strap.

6. The method according to claim 5, wherein the soft material comprises cotton.

7. The method according to claim 5, further comprising securing a medical device to the patient by laying at least one securement strap comprising a Velcro hook over the medical device and attaching the securement strap to the torso strap using the Velcro hook.

8. The method according to claim 5, wherein the medical device comprises tubes or catheters.
